(19) United States
(12) Patent Application Publication
(10) Pub. No.: US 2016/0168061 A1
(43) Pub. Date: Jun. 16, 2016

(54) METHODS AND APPARATUSES FOR GENERATING A POLYOL FROM BIOMASS USING MULTIPLE REACTION ZONES AND CATALYSTS

(71) Applicant: UOP LLC, Des Plaines, IL (US)

(72) Inventors: Tom N. Kalnes, LaGrange, IL (US); John Qianjun Chen, Glenview, IL (US); Joseph Anthony Kocal, Glenview, IL (US)

(21) Appl. No.: 14/566,206

(22) Filed: Dec. 10, 2014

(51) Int. Cl. C07C 29/132; B01J 8/24; B01J 8/20

(52) U.S. Cl. CPC C07C 29/132; B01J 8/20; B01J 8/24; B01J 2208/00752; B01J 2208/00893

(57) ABSTRACT

Methods and apparatuses for catalytically generating a polyol from biomass are provided. An exemplary method includes the steps of: contacting a feed stream comprising biomass to acid conditions in a first reaction zone, wherein the acid conditions in the first reaction zone are sufficient to hydrolyze a saccharide from the biomass to generate glucose; directing at least a first portion of a first effluent from the first reaction zone to a second reaction zone; and contacting the at least first portion of the first effluent with a first saccharide-to-polyol catalyst system under conditions suitable for catalytic conversion of saccharide to polyol.

### TECHNICAL FIELD

The technical field generally relates to methods and apparatuses for generating a polyol from biomass, and more particularly to methods and apparatuses for generating a polyol from biomass with using multiple reaction zones and catalysts.

### BACKGROUND

Polyols are valuable materials with uses such as preparation of purified terephthalic acid (PTA)/polyethylene terephthalate polymers (PET), cold weather fluids, cosmetics and many others. Generating polyols from cellulose instead of olefins can be a more environmentally friendly and economically attractive process. Previously, polyols have been generated from polyhydroxy compounds. Polyols have also been catalytically generated directly from cellulose in batch type and continuous processes. Catalytic conversion of cellulose into ethylene glycol over supported carbide catalysts, such as tungsten carbide catalysts, also has been achieved. However, the generation of polyols such as ethylene glycol from biomass conventionally has taken place in a single reactoi zone that utilizes a single catalyst. However, given the complexity of the processes involved in converting biomas to polyols, a single catalyst coupled with a single set of operating conditions does not allow for optimizing conversion to and/or selectivity of desired products and co-products.

Accordingly, it is desirable to provide methods and apparatuses for catalytic polyol production from biomass that utilize a plurality of reaction zones, each with a catalyst system and each capable of being operated under different operating conditions. Furthermore, other desirable features and characteristics of the present invention such as simpler product separation will become apparent from the subsequent detailed description of the invention and appended claims, taken in conjunction with the accompanying drawings and this background of the invention.

### BRIEF SUMMARY

Methods and apparatuses for catalytically generating polyols from biomass are provided. An exemplary method includes the steps of: contacting a feed stream comprising biomass to acid conditions in a first reaction zone, wherein the acid conditions in the first reaction zone are sufficient to hydrolyze a saccharide from the biomass to generate glucose. In this method, at least a first portion of a first effluent from the first reaction zone is directed to a second reaction zone, where the at least first portion of the first effluent is contacted with a first saccharide-to-polyol catalyst system under conditions suitable for catalytic conversion of saccharide to polyol.

In another embodiment, an exemplary method of generating polyols from biomass is provided. In this embodiment, the method comprises the steps of: contacting a feed stream comprising biomass to acid conditions in a first reaction zone, wherein the acid conditions in the first reaction zone are sufficient to hydrolyze a saccharide from the biomass to generate glucose; directing a first portion of a first effluent from the first reaction zone to a second reaction zone; contacting the first portion of the first effluent with a first saccharide-to-polyol catalyst system under conditions suitable for catalytic conversion of saccharide to polyol and generating a first product polyol stream, wherein the first saccharide-to-polyol catalyst system comprises a saccharide-to-polyol catalyst system that catalyzes a hydrogenation reaction; directing a second portion of the first effluent from the first reaction zone to a third reaction zone; and contacting the second portion of the first effluent with a second saccharide-to-polyol catalyst system under conditions suitable for catalytic conversion of saccharide to polyol and generating a second product polyol stream, wherein the second saccharide-to-polyol catalyst system comprises a saccharide-to-polyol catalyst system that catalyzes a retro-aldol reaction; wherein the first product polyol stream and the second product polyol stream are different, and the composition of polyols catalytically generated from the biomass is based on the relative amounts of first effluent directed to the second and third reaction zones.

In other embodiment, an exemplary apparatus for the catalytic generation of polyol from biomass comprises: a first reaction zone configured to receive a feed stream comprising biomass, subject the feed stream to conditions suitable for acid catalyzed hydrolysis of a saccharide in the feed stream to generate glucose, and provide a first effluent comprising glucose, wherein the first reaction zone is not configured to receive a hydrogen stream; and a second reaction zone in fluid communication with the first reaction zone, the second reaction zone configured to receive and contact at least a portion of the first effluent with a hydrogen stream and a first saccharide-to-polyol catalyst system under conditions suitable for conversion of glucose to a polyol.

### BRIEF DESCRIPTION OF THE DRAWING

The various embodiments will hereinafter be described in conjunction with the following drawing figure, wherein:

FIG. 1 is a block diagram of an apparatus comprising first and second reaction zones for catalytic polyol generation from biomass in accordance with an exemplary embodiment described herein.

FIG. 2 is a block diagram of an apparatus comprising first, second, and third reaction zones for catalytic polyol generation from biomass in accordance with an exemplary embodiment described herein.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

Various embodiments provided herein relate to methods and apparatuses for the catalytic generation of at least one polyol from a feedstock comprising a biomass, in some embodiments, whole biomass, and in some particular embodiments, whole cellulostic biomass. As used herein, the term “cellulostic biomass” refers to any biologically derived or waste derived material that contains cellulose. Cellulostic biomass is typically derived from plant matter and contains three major polysaccharides, cellulose, pectin, and hemicellulose. Polymeric lignin is also typically present in significant amounts. Further, impurities, such as metals and ash, are typically present as well. Cellulostic biomass used in methods and apparatuses described herein may be whole biomass; that is, cellulostic biomass used in methods and apparatuses described herein may containing cellulose, hemicellulose, and polymeric lignin. In some embodiments, the feedstock comprises waste cellulose, such as waste cellulose typically found in municipal or industrial wastewater. Further, in some embodiments, the feedstock may contain any desired combination of biomass and waste cellulose, or cellulose from any other source.

Conventional methods and apparatuses for the catalytic generation of polyols from biomass rely on reactions that occur between a saccharide, water and hydrogen. As used herein, the term “saccharide” is meant to include any of monosaccharides, disaccharides, oligosaccharides, and polysaccharides. A saccharide may be edible, inedible, amorphous, or crystalline in nature. Polysaccharides may consist of one or more monosaccharides linked by glycosidic bonds. Examples of polysaccharides include glycogen, cellulose, hemicellulose, starch, chitin, and combinations thereof.

Conducting catalytic generation of polyols from saccharides generally relies on multiple reactions, including hydrolysis, hydrogenolysis, retro-aldol and hydrogenation reactions. In particular, polysaccharides in biomass are broken down into saccharides with a lower molecular weight than the starting polysaccharides via hydrolysis reactions. In some embodiments, the lower molecular weight saccharides (i.e., the intermediate saccharides) may include one or more monosaccharides, one or more disaccharides, one or more trisaccharides, and mixtures thereof. The intermediate saccharides may then be converted to polyols (e.g., ethylene glycol or C4 (four carbon) polyols) via a number of different reaction pathways. For instance, polysaccharides in biomass may be broken down into glucose, which may then be subjected to hydrogenation to generate mannitol and sorbitol, which further undergo catalytic hydrogenolysis to generate C4 polyols. An alternative reaction pathway for the treatment of glucose is catalytic retro-aldol conversion to erythrose and glycolaldehyde. Glycolaldehyde may then be converted into ethylene glycol via hydrogenation. Erythrose may be subject to a further retro-aldol conversion to generate glycolaldehyde, which may be converted into ethylene glycol via hydrogenation. Alternatively or in addition, at least a portion of the erythrose may be converted to C4 polyols via hydrogenation. Hydrogenation of glycoaldehyde to ethylene glycol may be conventionally catalyzed, for example with certain noble metal catalyst systems that are active in and resilient to aqueous environments.

The various reaction pathways described above are optimally conducted with different catalyst materials under different conditions. For instance, hydrolysis reactions that break down polysaccharides into glucose are conventionally catalyzed under acidic conditions. Catalytic retro-aldol conversion of glucose to erythrose and glycolaldehyde may be catalyzed, for example, with a conventional tungsten/activated carbon catalysts. Hydrogenation of glucose to mannitol and sorbitol may be catalyzed, for example, with conventional noble metal catalyst systems. Further, optimal conditions for any of these catalytically driven processes may not be optimal for a subsequent step of hydrogenolysis and/or hydrogenation to convert the catalytically derived products to valuable product polyols (including ethylene glycol). Additionally, any one of the reaction pathways outlined above may be preferred at one time over another due to variances in biomass feed composition, or preference of different products and co-products that may be preferentially generated via one pathway over another.

Further, in some instances, the catalytic generation of polyols from biomass via a conventional single reactor process generates undesirable co-products that are difficult to separate from desired polyols, and/or commercially valuable reaction intermediates (i.e., desirable hydrolysis co-products) that may not survive the complete reaction process. In such instances it would be beneficial to conduct catalytic generation of polyols from saccharides in stages, e.g., conduct hydrolysis separately from hydrogenolysis and hydrogenation reactions, thereby allowing for separation and collection of undesirable hydrolysis co-products and/or desirable hydrolysis co-products before subjecting the co-products to hydrogenolysis and hydrogenation. Other benefits, such as reduction of energy input by reducing recycle stream volumes, may also be realized by utilization of a plurality of reaction zones, each with a different catalyst.

As such, methods and apparatuses provided herein utilize a plurality of reaction zones, each with a different catalyst system, to allow for selection of improved conditions for each of the selected catalysts, and/or process control to select for increased production of preferred products and/or co-products. The plurality of reaction zones includes a first reaction zone that receives an input stream comprising one or more polysaccharides (from biomass) and subjects the input stream to a first set of reaction conditions to hydrolyze the polysaccharides to glucose. This first set of reaction conditions conventionally includes contacting the input stream with an acidic aqueous environment without hydrogen input. A first effluent from the first reaction zone thus includes glucose in an aqueous environment. At least a portion of this first effluent is directed to a second reaction zone where glucose is contacted with a catalyst system and hydrogen so as to conduct catalyzed hydrogenation reactions or catalyzed retro-aldol reactions.

In some embodiments, at least a portion of the first effluent may be sent to a first separation zone prior to being directed to the second reaction zone. This first separation zone may be configured to receive the at least portion of the first effluent and separate at least a first hydrolysis co-product stream comprising an undesirable hydrolysis co-product and/or a desirable hydrolysis co-product. After this separation, the remainder of the at least portion of first effluent sent to the first separation zone may be directed to one or more reaction zones, as provided herein.

In some embodiments, the first effluent may be divided and sent to a plurality of reaction zones in parallel, such as a second reaction zone and a third reaction zone in parallel, where each reaction zone contacts a portion of the first effluent with a different catalyst system. In such embodiments, each reaction zone may be operated independently under conditions appropriate for catalysts contained therein. Such embodiments also allow for user selection of the proportion of first effluent that is directed to each of the plurality of reaction zones. In this way, a proportion of products and co-products that are preferentially generated via each pathway may be selected over products and co-products that are preferentially generated via another.

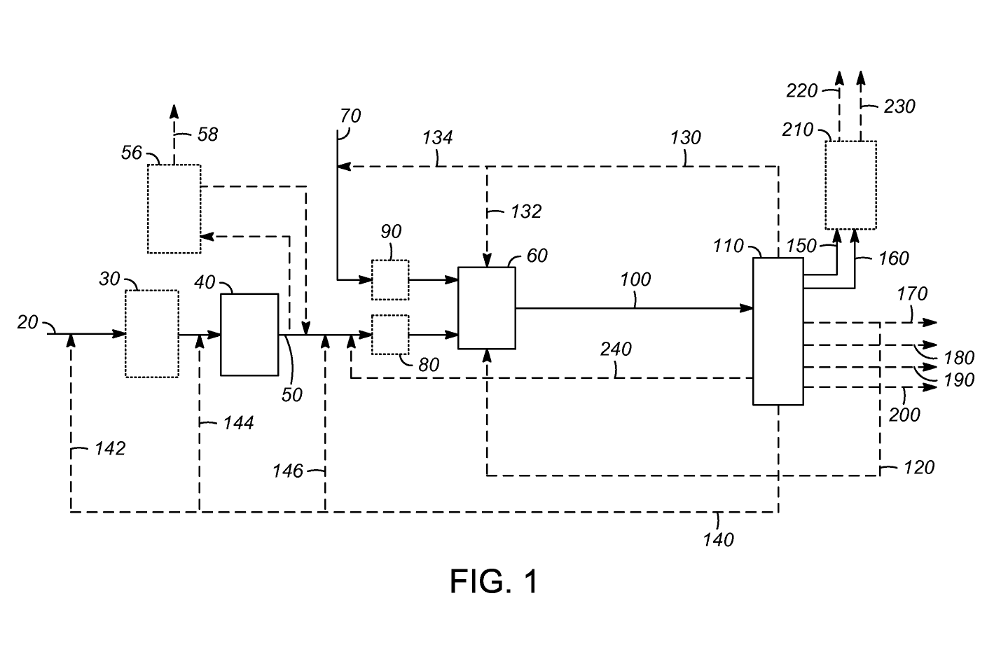

In some embodiments, the first effluent may be sent to a plurality of reaction zones in series, such as a second reaction zone followed by a third reaction zone, where each reaction zone contacts at least a portion of an effluent from a preceding reaction zone with a different catalyst. In such embodiments, each reaction zone in sequence may be operated independently under conditions appropriate for catalysts contained therein. Further, in such embodiments, process conditions for each of the plurality of reaction zones, such as residence time, may be controlled so as to allow for adjustment of the proportion of products and co-products that are preferentially generated in each reaction zone. An exemplary method will now be described with reference to FIG. 1 depicting the flow scheme of a first exemplary apparatus having at least a first reaction zone where polysaccharides are catalytically converted to glucose without providing hydrogen gas to the first reaction zone, and a second reaction zone where glucose is catalytically converted to a polyol, such as ethylene glycol, with hydrogen provided. The exemplary method begins with an input stream 20 comprising biomass. One challenge in processing biomass to generate polyols is that biomass, such as whole cellulostic biomass, often is in a solid state. Therefore, pretreatment of the biomass may be performed in order to facilitate continuous transporting. In some embodiments, pretreatment operations include sizing, drying, grinding, and combinations thereof. In some exemplary embodiments, biomass, including whole cellulostic biomass, may be processed by one or more pretreatment techniques into solid particles of a size that may be passed or moved through a continuous process using a liquid or gas flow, or mechanical processing as part of the input stream into a first reaction zone. In some embodiments, one or more pretreatments may be utilized to reduce or eliminate various components, such as polymerized lignin, from the input stream 20. For instance, in some embodiments, an amount of polymerized lignin present in the input stream 20 is reduced (e.g., converted to depolymerized lignin) via hot water treatment, steam treatment, hydrolysis, pyrolysis, thermal treatment, chemical treatment, biological treatment, catalytic treatment, and combinations thereof.

Thus, in some embodiments and as shown in FIG. 1, the input stream 20 is optionally subjected to a pretreatment process comprising sizing, grinding, drying, hot water treatment, steam treatment, hydrolysis, pyrolysis, thermal treatment, chemical treatment, biological treatment, catalytic treatment, pressure treatment, or a combination thereof, in an optional pretreatment zone 30. In some embodiments, pressure treatment comprises treating a feedstock with a protocol that includes a pressurization and/or depressurization step. For instance, in some embodiments, a pressure treatment comprises steam explosion, ammonia fiber explosion, hot hydrogen explosion, or the like. In some embodiments, a pressure treatment is used together with a thermal or chemical treatment.

At least a portion of input stream 20 is introduced into a first reaction zone 40, where polysaccharides from the biomass are catalytically converted, such as under conventional acid catalyzed hydrolysis conditions (e.g., in an acidic aqueous environment at a pH of about 2.0 to about 6.5, a temperature of about 20° C. to about 350° C., and a pressure of about 15 psig to about 1000 psig) to generate glucose.

The first reaction zone 40 generates a first reaction zone effluent 50 containing glucose. At least a portion of the first reaction zone effluent 50 may be directed to an optional first separation zone 56 where a hydrolysis co-product stream 58 is separated. The first reaction zone effluent 50 is then directed to a second reaction zone 60, which is configured to receive the first reaction zone effluent 50 and combine the first reaction zone effluent 50 with one or more additional constituents (e.g., water and/or hydrogen) to generate a reaction mixture. The reaction mixture is contacted with a catalyst system under conditions suitable for the catalytic conversion of glucose to a polyol (and in some particular embodiments, ethylene glycol). In some methods and as shown in FIG. 1, the first reaction zone effluent 50 is provided to the second reaction zone 60 separately from a hydrogen stream 70.

Depending on what, if any, pretreating steps are employed, water may already be present in the first reaction zone effluent 50. Further, in some embodiments, water may be added to the first reaction zone effluent 50, the hydrogen stream 70, or both, or water may be introduced to the second reaction zone 60 via an independent water stream (not shown in FIG. 1). In some embodiments, the hydrogen stream 70 may be heated in an optional first hydrogen processing zone 90 prior to introduction to the second reaction zone 60. For instance, in some embodiments, hydrogen stream 70 may be heated to a temperature in excess of the reaction temperature of the second reaction zone 60. In some embodiments, the hydrogen stream 70 is heated to a temperature such that when the hydrogen stream 70 is introduced into a second reactor (within the second reaction zone 60) and combined with the first reaction zone effluent 50, the temperature of the resulting reaction mixture is at or above the necessary reaction temperature for a selected glucose-to-polyol catalyst system. In particular, this allows for methods in which the temperature of the first reaction zone effluent 50 is controlled so as not to exceed the decomposition temperature of cellulose and/or so as not to exceed the charring temperature of cellulose, while still allowing a mixture of the first reaction zone effluent 50 and the hydrogen stream 70 to be at or above a necessary reaction temperature in the second reaction zone 60. Specifically, hydrogen stream 70 may be heated to a temperature in excess of the reaction temperature of the second reaction zone 60 such that when hydrogen stream 70 and first reaction zone effluent 50 are introduced into the second reaction zone 60 and mixed, the resulting reaction mixture is at or above the necessary reaction temperature for glucose-to-polyol conversion. Thus, the temperature of the first reaction zone effluent 50 may be controlled so as not to exceed the decomposition temperature of glucose or so as not to exceed the charring temperature of glucose.

Further, in some embodiments, the first reaction zone effluent 50 or the hydrogen stream 70 may be pressurized to a reaction pressure in an optional first glucose processing zone 80 or the optional first hydrogen processing zone 90, respectively, before being introduced into the second reaction zone 60. In some embodiments, the first reaction zone effluent 50 and the hydrogen stream 70 are both pressurized to a reaction pressure. In some embodiments, a reaction pressure may be about 15 psig to about 2500 psig, such as about 15 psig to about 1000 psig.

In some embodiments, the saccharide-to-polyol catalyst system may reside within the second reaction zone 60. In other embodiments, the saccharide-to-polyol catalyst system may continuously or intermittently pass through the second reaction zone 60. In some embodiments, the second reaction zone 60 includes a conventional reactor system. Exemplary suitable reactor systems include ebullating catalyst bed systems, immobilized catalyst reaction systems having catalyst channels, augured reaction systems, fluidized bed reactor systems, mechanically mixed reaction systems, or slurry reactor systems, also known as three phase bubble column reactor systems. In some specific embodiments, the second reaction zone 60 employs a slurry reactor system. In such embodiments, the saccharide-to-polyol catalyst system may be mixed with the first reaction zone effluent 50 and a sufficient amount of water to form a slurry prior to introduction into the slurry reactor. Catalytic conversion occurs within the slurry reactor and the catalyst system is transported out of the reactor as a component in the second effluent 100. The slurry reactor system may be operated at temperatures from about 100° C. to about 350° C. and the hydrogen pressure may be greater than about 150 psig. In some embodiments, the temperature in the slurry reactor system may range from about 150° C. to about 350° C., such as about 200° C. to about 280° C. In some embodiments, the slurry reactor is operated with a water to saccharide weight ratio of about 1:100 or greater, such as about 1:20 or greater, such as about 1:5 or greater. In some embodiments, the slurry reactor is operated with a catalyst to saccharide weight ratio of about 1:500 or greater, such as about 1:200 or greater, such as about 1:100 or greater, such as about 1:10 or greater. In some embodiments, the slurry reactor is operated with a pH of less than about 10. In some embodiments, the slurry reactor is operated with a residence time of greater than 5 minutes. In some embodiments, the slurry reactor is operated such that any combination of these operating conditions are met.

In a specific exemplary embodiment, the slurry reactor is operated with a water to saccharide weight ratio of about 1:20 or greater and with a catalyst to saccharide weight ratio of about 1:100 or greater. In yet another specific exemplary embodiment, the slurry reactor is operated with a water to saccharide weight ratio of about 1:5 or greater and with a catalyst to saccharide weight ratio of about 1:10 or greater.

In some embodiments, a saccharide-to-polyol catalyst system comprises an elemental noble metal, i.e., ruthenium (Ru), rhodium (Rh), palladium (Pd), silver (Ag), osmium (Os), iridium (Ir), platinum (Pt), and gold (Au). In some specific embodiments, a saccharide-to-polyol catalyst system comprises at least two active metal components: a first elemental metal component of platinum (Pt), palladium (Pd), ruthenium (Ru), or a combination thereof; and a second metal component of molybdenum (Mo), tungsten (W), vanadium (V), nickel (Ni), cobalt (Co), iron (Fe), tantalum (Ta), niobium (Nb), titanium (Ti), chromium (Cr), zirconium (Zr), or any combination thereof, wherein the second metal component is in the elemental state, a carbide compound, a nitride compound, a phosphide compound, or any combination thereof. The saccharide-to-polyol catalyst system may further comprise a support material which can be a powder, or formed in specific shapes such as spheres, extrudates, pills, pellets, tablets, irregularly shaped particles, monolithic structures, catalytically coated tubes, or catalytically coated heat exchanger surfaces. Examples of suitable support materials include the refractory inorganic oxides including but not limited to silica, alumina, silica-alumina, titania, zirconia, magnesia, clays, zeolites, molecular sieves, etc. As will be understood, silica-alumina is not a mixture of silica and alumina but rather is an acidic and amorphous material that has been cogelled or coprecipitated. Carbon and activated carbon may also be employed as support materials. Specific suitable supports include carbon, Al₂O₃, ZrO₂, SiO₂, MgO, CeₓZrO_{y}, TiO₂, SiC. Mixtures of any of these or any other suitable support materials may also be used.

Alternatively or in addition, compound catalyst systems such as those described in U.S. Pat. No. 8,222,462 and U.S. Pat. No. 8,222,463 may be used for saccharide to polyol catalytic conversion. For instance, a compound catalyst system comprising an unsupported component comprising a compound selected from the group consisting of a tungsten compound, a molybdenum compound, and any combination thereof, and a supported component comprising an active metal component selected from the group consisting of Pt, Pd, Ru, Rh, Ni, Ir, and combinations thereof on a solid catalyst support. Suitable solid catalyst supports include those provided above.

In some embodiments, certain components of a catalyst system may be soluble in water. If catalyst recovery and/or recycle is desired in such embodiments, conventional techniques for recovery and/or recycling of water soluble catalyst components may be used.

Within the second reaction zone 60 and at operating conditions as described above, the reactants (i.e., saccharide, hydrogen, and water) proceed through catalytic conversion reactions to produce at least one polyol in a second effluent 100. In some embodiments, the product polyol includes ethylene glycol, propylene glycol, or a combination thereof. In some embodiments, at least one co-product is also produced. Such co-products may include alcohols, organic acids, aldehydes, monosaccharides, polysaccharides, phenolic compounds, hydrocarbons, glycerol, depolymerized lignin, carbohydrates, and proteins. In some embodiments, a plurality of co-products may be produced. Some of the co-products may have commercial value and may be isolated and recovered in addition to the product polyols. Co-products may also be reaction intermediates which, in some embodiments, are separated from the second reaction zone effluent and recycled back to the second reaction zone.

Unreacted hydrogen, water, and saccharides may also be present in the second effluent 100 along with co-products. In some embodiments, unreacted hydrogen, water, saccharides, or any combination thereof, are separated in a product recovery zone 110 and recycled back to the second reaction zone 60. In some embodiments, hydrogen is separated from the second effluent 100 to generate a hydrogen recycle stream 130 before water is separated from the second effluent 100 to generate a water recycle stream 140. The hydrogen recycle stream 130 may be recycled to one or more of a number of different locations within the process depending upon the specific embodiment employed. For example, at least a first portion 132 of hydrogen recycle stream 130 may be recycled to the second reaction zone 60. Alternatively or in addition, at least a second portion 134 of hydrogen recycle stream 130 may be combined with fresh hydrogen or make-up hydrogen (e.g., as part of the hydrogen stream 70) before being introduced into second reaction zone 60. In some embodiments, the hydrogen recycle stream 130 is purified (not shown) before recycling. In some embodiments, a gas-liquid separator is used to separate the hydrogen from the second effluent 100.

Similarly, water from the second effluent 100 may be separated via the product recovery zone 110 to form the water recycle stream 140 that may be recycled to one or more of a number of different locations within the process depending upon the specific embodiment employed. For example, in some embodiments at least a portion of the water recycle stream 140 is recycled to combine with the biomass containing input stream 20 prior to the optional preprocessing zone 30 via a first water recycle stream portion 142, between the optional preprocessing zone 30 and first reaction zone 40 via a second water recycle stream portion 144, or between the first reaction zone 40 and second reaction zone 60 via a third water recycle stream portion 146. Further, in some embodiments the water recycle stream 140 may be purified via any suitable water purification system (not shown in FIG. 1) before being recycled.

In embodiments of the methods described herein, one or more product polyols are separated from the second effluent 100 in the product recovery zone 110. Multiple product polyol streams may be generated in the product recovery zone 110. For instance, one or more polyol streams may include an ethylene glycol stream 150 and/or a propylene glycol stream 160.

In some embodiments, co-products may also be separated into one or more co-product streams, and optionally recycled back to the second reaction zone 60 as optional co-product recycle stream 120. In some embodiments, one or more co-product streams may include a low molecular weight co-product stream 170 comprising one or more co-products having a molecular weight lower than ethylene glycol, such as alcohols. In some embodiments, one or more co-product streams may include a high molecular weight co-product stream 180 comprising one or more co-products having a molecular weight higher than propylene glycol, such as glycerol. In some embodiments, one or more co-product streams may include a fuel gas stream 190. In some embodiments, one or more co-product streams may include a non-volatile residue stream 200. Additionally, apparatuses described herein may further comprise a product purification zone 210, where one or more product polyol streams are purified to generate one or more high purity polyol streams 220 and 230 by any suitable conventional purification technique.

Depending upon the particulars of the saccharide-to-polyol catalyst system, the product recovery zone 110 may also separate catalyst particles from the second effluent 100. In some embodiments, catalyst particles 240 are separated from the second effluent 100 either before or after recovery of products and/or co-products. Catalyst particles may be separated from the second effluent 100 using one or more techniques such as direct filtration, settling followed by filtration, hydrocyclone, fractionation, centrifugation, the use of flocculants, precipitation, extraction, evaporation, or combinations thereof. In some embodiments, catalyst particles 240 are separated from the second effluent 100 after generation of hydrogen recycle stream 130 but before generation of water recycle stream 140. In some embodiments and as seen in FIG. 1, separated catalyst particles 240 are recycled to the second reaction zone 60. In some related embodiments, separated catalyst particles are reactivated before being recycled to the second reaction zone 60 (not shown in FIG. 1).

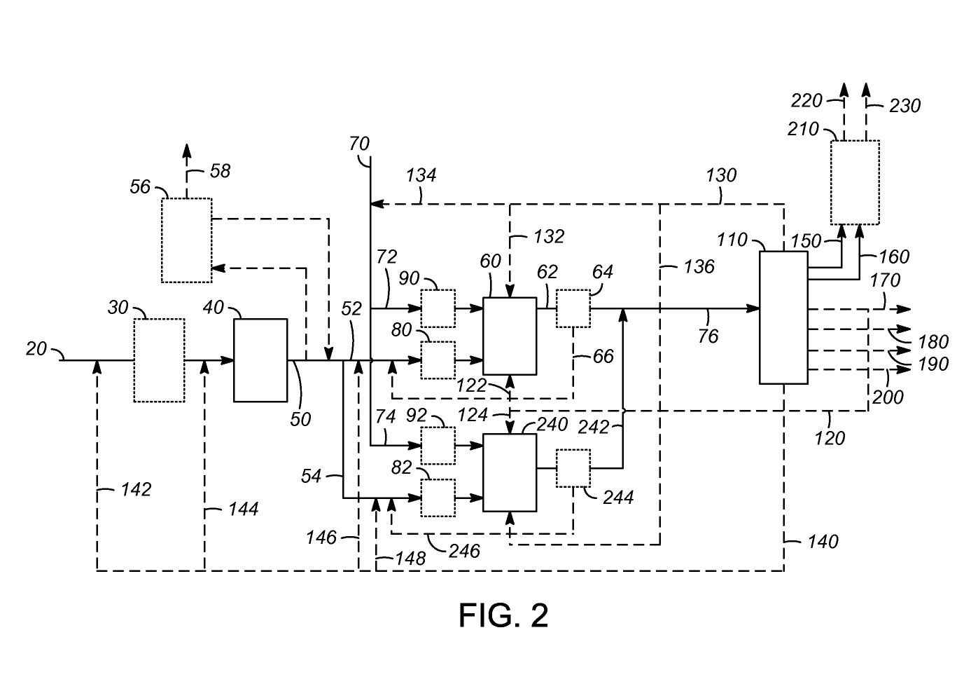

A second exemplary method will now be described with reference to FIG. 2 depicting the flow scheme of a second exemplary apparatus having at least a first reaction zone where polysaccharides are catalytically converted to glucose without providing hydrogen gas to the first reaction zone, and having second and third reaction zones where glucose is catalytically converted to a polyol, such as ethylene glycol, with hydrogen provided. This embodiment is similar to that described above with reference to FIG. 1, but in this embodiment, portions of the effluent from the first reaction zone are directed to second and third reaction zones, which are located in parallel and each contact their respective portions of the effluent from the first reaction zone with a different catalyst.

Again, this exemplary embodiment begins with an input stream 20 comprising biomass. As described above and shown in FIG. 2, the input stream 20 is optionally subjected to a pretreatment process comprising sizing, grinding, drying, hot water treatment, steam treatment, hydrolysis, pyrolysis, thermal treatment, chemical treatment, biological treatment, catalytic treatment, or a combination thereof, in an optional pretreatment zone 30. At least a portion of input stream 20 is introduced into a first reaction zone 40, where polysaccharides from the biomass are catalytically converted, such as under conventional acid catalyzed hydrolysis conditions (e.g., in an aqueous environment) to generate glucose.

The first reaction zone 40 thus generates a first reaction zone effluent 50 containing glucose. At least a portion of the first reaction zone effluent 50 may be directed to an optional first separation zone 56 where a hydrolysis co-product stream 58 is separated. A first portion 52 of the first reaction zone effluent 50 is directed to a second reaction zone 60, which is configured to receive the first portion 52 and combine the first portion 52 with one or more additional constituents (e.g., water and/or hydrogen) to generate a reaction mixture. The reaction mixture is then contacted with a first catalyst under conditions suitable for the catalytic conversion of glucose to a polyol (and in some particular embodiments, ethylene glycol). In some methods and as shown in FIG. 2, the first portion 52 is provided to the second reaction zone 60 separately from a first hydrogen stream 72.

A second portion 54 of the first reaction zone effluent 50 is directed to a third reaction zone 240, which is configured to receive the second portion 54 and combine the second portion 54 with one or more additional constituents (e.g., water and/or hydrogen) to generate a second reaction mixture. The second reaction mixture is then contacted with a second catalyst under conditions suitable for the catalytic conversion of glucose to a polyol (and in some particular embodiments, ethylene glycol). In some methods and as shown in FIG. 2, the second effluent portion 54 is provided to the third reaction zone 240 separately from a second hydrogen stream 74.

In this embodiment, the catalyst system utilized in the first reaction zone differs from the catalyst system utilized in the second reaction zone 240. Configured as such, these apparatus allow for operation of the second and third reaction zones at different conditions (e.g., temperature, pressure, reagent and catalyst proportions, residence time, etc.) such that the operation of each reaction zone is tailored to the catalyst system contained therein. Note that various conventional catalyst systems may be used in each of the second and third reaction zones, and that different catalyst systems may be selected that result in production of different products and co-products. This selectivity, coupled with control of the relative proportions of the first effluent portion 52 and second effluent portion 54, provides a user the ability to modulate the identities and relative amounts of products and co-products derived from a particular feed.

As described above, water may already be present in the first effluent 50 depending on what, if any, pretreating steps are employed. In addition or in the alternative, in some embodiments, water is added to the first effluent portion 52, the first hydrogen stream 72, or both, or water may be introduced to the second reaction zone 60 via an independent water stream (not shown in FIG. 2). In some embodiments, the first hydrogen stream 72 may be heated in an optional first hydrogen processing zone 90 prior to introduction to the second reaction zone 60. For instance, in some embodiments, the first hydrogen stream 72 may be heated to a temperature in excess of the reaction temperature of the second reaction zone 60. In some embodiments, the first hydrogen stream 72 is heated to a temperature such that when the first hydrogen stream 72 is introduced into a second reactor (within the second reaction zone 60) and combined with the first effluent portion 52, the temperature of the resulting reaction mixture is at or above the necessary reaction temperature for a selected first glucose-to-polyol catalyst system. In particular, this allows for methods in which the temperature of the first effluent portion 52 is controlled so as not to exceed the decomposition temperature of glucose and/or so as not to exceed the charring temperature of glucose, while still allowing a mixture of the first effluent portion 52 and the first hydrogen stream 72 to be at or above a necessary reaction temperature in the second reaction zone 60. Specifically, first hydrogen stream 72 may be heated to a temperature in excess of the reaction temperature of the second reaction zone 60 such that when first hydrogen stream 72 and first effluent portion 52 are introduced into the second reaction zone 60 and mixed, the resulting reaction mixture is at or above the necessary reaction temperature for glucose to polyol conversion. Thus, the temperature of the first effluent portion 52 may be controlled so as not to exceed the decomposition temperature of glucose or so as not to exceed the charring temperature of glucose.

Likewise, water may also be added to the second effluent portion 54, the second hydrogen stream 74, or both, or water may be introduced to the third reaction zone 240 via an independent water stream (not shown in FIG. 2). In some embodiments, the second hydrogen stream 74 may be heated in an optional second hydrogen processing zone 92 prior to introduction to the third reaction zone 240. For instance, in some embodiments, the second hydrogen stream 74 may be heated to a temperature in excess of the reaction temperature of the third reaction zone 240. In some embodiments, the second hydrogen stream 74 is heated to a temperature such that when the second hydrogen stream 74 is introduced into a third reactor (within the third reaction zone 240) and combined with the second effluent portion 54, the temperature of the resulting reaction mixture is at or above the necessary reaction temperature for a selected second glucose-to-polyol catalyst system. In particular, this allows for methods in which the temperature of the second effluent portion 54 is controlled so as not to exceed the decomposition temperature of glucose and/or so as not to exceed the charring temperature of glucose, while still allowing a mixture of the second effluent portion 54 and the second hydrogen stream 74 to be at or above a necessary reaction temperature in the third reaction zone 240. Specifically, second hydrogen stream 74 may be heated to a temperature in excess of the reaction temperature of the third reaction zone 240 such that when second hydrogen stream 74 and second effluent portion 54 are introduced into the third reaction zone 64 and mixed, the resulting reaction mixture is at or above the necessary reaction temperature for glucose to polyol conversion. Thus, the temperature of the second effluent portion 54 may be controlled so as not to exceed the decomposition temperature of glucose or so as not to exceed the charring temperature of glucose.

The respective catalyst systems described above may reside within one or both of the second reaction zone 60 and third reaction zone 240. In other embodiments, the respective catalyst systems may continuously or intermittently pass through either or both of the second reaction zone 60 and third reaction zone 240. In some embodiments, the second reaction zone 60 and/or third reaction zone 240 include conventional reactor systems. Exemplary suitable reactor systems include ebullating catalyst bed systems, immobilized catalyst reaction systems having catalyst channels, augured reaction systems, fluidized bed reactor systems, mechanically mixed reaction systems, or slurry reactor systems, also known as three phase bubble column reactor systems.

In some specific embodiments, the second reaction zone 60 and/or third reaction zone 240 employ a slurry reactor system. As such, in some embodiments, a selected catalyst system may be mixed with the first effluent portion 52 and/or second effluent portion 54 and a sufficient amount of water to form a slurry prior to introduction into the respective slurry reactor. Catalytic conversion occurs within a slurry reactor and the catalyst system is transported out of the reactor as a component in the second effluent 62 and/or third effluent 240. The slurry reactor system may be operated at temperatures from about 100° C. to about 350° C. and the hydrogen pressure may be greater than about 150 psig. In some embodiments, the temperature in a slurry reactor may range from about 150° C. to about 350° C., such as about 200° C. to about 280° C. In some embodiments, the slurry reactor is operated with a water to saccharide weight ratio of about 1:100 or greater, such as about 1:20 or greater, such as about 1:5 or greater. In some embodiments, the slurry reactor is operated with a catalyst to saccharide weight ratio of about 1:200 or greater, such as about 1:100 or greater, such as about 1:10 or greater. In some embodiments, the slurry reactor is operated with a pH of less than about 10. In some embodiments, the slurry reactor is operated with a residence time of greater than 5 minutes. In some embodiments, the slurry reactor is operated such that any combination of these operating conditions are met.

In a specific exemplary embodiment, the slurry reactor is operated with a water to saccharide weight ratio of about 1:20 or greater and with a catalyst to saccharide weight ratio of about 1:100 or greater. In yet another specific exemplary embodiment, the slurry reactor is operated with a water to saccharide weight ratio of about 1:5 or greater and with a catalyst to saccharide weight ratio of about 1:10 or greater.

In some embodiments, a saccharide-to-polyol catalyst system comprises an elemental noble metal, i.e., ruthenium (Ru), rhodium (Rh), palladium (Pd), silver (Ag), osmium (Os), iridium (Ir), platinum (Pt), and gold (Au). In some specific embodiments, a saccharide-to-polyol catalyst system comprises at least two active metal components: a first elemental metal component of platinum (Pt), palladium (Pd), ruthenium (Ru), or a combination thereof; and a second metal component of molybdenum (Mo), tungsten (W), vanadium (V), nickel (Ni), cobalt (Co), iron (Fe), tantalum (Ta), niobium (Nb), titanium (Ti), chromium (Cr), zirconium (Zr), or any combination thereof, wherein the second metal component is in the elemental state, a carbide compound, a nitride compound, a phosphide compound, or any combination thereof. In some embodiments, such catalyst systems may lead to catalytic conversion of glucose-to-polyols via conventional hydrogenation.

In some embodiments, a saccharide-to-polyol catalyst system comprises a nickel tungsten carbide catalyst. In some embodiments, such catalysts systems may lead to retro-aldol conversion of glucose to ethylene glycol and other polyols via intermediates such as erythrose and glycolaldehyde.

In some embodiments, a saccharide-to-polyol catalyst system may further comprise a support material which can be a powder, or formed in specific shapes such as spheres, extrudates, pills, pellets, tablets, irregularly shaped particles, monolithic structures, catalytically coated tubes, or catalytically coated heat exchanger surfaces. Examples of suitable support materials include the refractory inorganic oxides including but not limited to silica, alumina, silica-alumina, titania, zirconia, magnesia, clays, zeolites, molecular sieves, etc. As will be understood, silica-alumina is not a mixture of silica and alumina but rather is an acidic and amorphous material that has been cogelled or coprecipitated. Carbon and activated carbon may also be employed as support materials. Specific suitable supports include carbon, Al₂O₃, ZrO₂, SiO₂, MgO, CeₓZrO_{y}, TiO₂, SiC. Mixtures of any of these or any other suitable support materials may also be used.

In some embodiments, the catalyst system utilized in the second reaction zone 60 comprises a hydrolysis catalyst, e.g., a noble metal catalyst described above. In some embodiments, the catalyst system utilized in the third reaction zone 240 comprises a retro-aldol catalyst, e.g., a nickel tungsten carbide catalyst described above. Thus, within the second reaction zone 60 and third reaction zone 240 and at operating conditions, the reactants (i.e., saccharide, hydrogen, and water) proceed through catalytic conversion reactions to produce at least one polyol in a second effluent 62 and at least one polyol in a third effluent 242. In some embodiments, the product polyol from the second reaction zone 60 and/or third reaction zone 240 includes ethylene glycol, propylene glycol, or a combination thereof. In some embodiments, at least one co-product is also produced in one or both of the second effluent 62 and third effluent 242. Such co-products may include alcohols, organic acids, aldehydes, monosaccharides, polysaccharides, phenolic compounds, hydrocarbons, glycerol, depolymerized lignin, carbohydrates, and proteins. In some embodiments, a plurality of co-products may be produced. Some of the co-products may have commercial value and may be isolated and recovered in addition to the product polyols. Co-products may also be reaction intermediates which, in some embodiments, are separated from the second effluent 62 and/or third effluent 242 and recycled back to the appropriate reaction zone. In embodiments, utilization of different catalyst systems in the second reaction zone 60 and third reaction zone 240 leads to generation of different product and/or co-product streams from each of the reaction zones.

Unreacted hydrogen, water, and saccharides may also be present in the second effluent 62 and/or third effluent 242 along with co-products. In some embodiments, unreacted hydrogen, water, saccharides, or any combination thereof, are separated in a product recovery zone 110 and recycled back to the second reaction zone 60 and/or third reaction zone 240. In some embodiments, hydrogen is separated to generate a hydrogen recycle stream 130 before water is separated to generate a water recycle stream 140. The hydrogen recycle stream 130 may be recycled to one or more of a number of different locations within the process depending upon the specific embodiment employed. For example, at least a first portion 132 of hydrogen recycle stream 130 may be recycled to the second reaction zone 60. Alternatively or in addition, at least a second portion 134 of hydrogen recycle stream 130 may be combined with fresh hydrogen or make-up hydrogen (e.g., as part of the hydrogen stream 70) before being introduced into second reaction zone 60 and/or third recycle zone 240. Further, at least a third portion 136 of hydrogen recycle stream 130 may be recycled to the third reaction zone 240. In some embodiments, the hydrogen recycle stream 130 is purified (not shown) before recycling. In some embodiments, a gas-liquid separator is used to separate the hydrogen.

Similarly, water from the second effluent 62, third effluent 242, or a combination thereof 76 may be separated via the product recovery zone 110 to form a water recycle stream 140 that may be recycled to one or more of a number of different locations within the process depending upon the specific embodiment employed. For example, in some embodiments at least a portion of a water recycle stream 140 is recycled to combine with the biomass containing input stream 20 prior to the optional preprocessing zone 30 via a first water recycle stream portion 142, between the optional preprocessing zone 30 and first reaction zone 40 via a second water recycle stream portion 144, between the first reaction zone 40 and second reaction zone 60 via a third water recycle stream portion 146, and/or between the first reaction zone 40 and third reaction zone 240 via a fourth water recycle stream portion 148. Further, in some embodiments the water recycle stream 140 may be purified via any suitable water purification system (not shown in FIG. 1) before being recycled.

In embodiments of the methods described herein, one or more product polyols are separated from the second effluent 62, third effluent 242, or a combination thereof. In some embodiments and as seen in FIG. 2, the one or more product polyols are separated from combined effluent 76 in a product recovery zone 110. Multiple product polyol streams may be generated by the product recovery zone 110. For instance, one or more polyol streams may include an ethylene glycol stream 150 and/or a propylene glycol stream 160.

In some embodiments, co-products may also be separated into one or more co-product streams, and optionally recycled back to the second reaction zone 60, the third reaction zone 240, or both as optional co-product recycle stream 120. For instance, at least a first portion of the optional co-product recycle stream 120 may be recycled back to the second reaction zone 60 as a co-product recycle stream portion 122, and/or at least a second portion of the optional co-product recycle stream 120 may be recycled back to the third reaction zone 240 as a co-product recycle stream portion 124. In some embodiments, one or more co-product streams may include a low molecular weight co-product stream 170 comprising one or more co-products having a molecular weight lower than ethylene glycol, such as alcohols. In some embodiments, one or more co-product streams may include a high molecular weight co-product stream 180 comprising one or more co-products having a molecular weight higher than propylene glycol, such as glycerol. In some embodiments, one or more co-product streams may include a fuel gas stream 190. In some embodiments, one or more co-product streams may include a non-volatile residue stream 200. Additionally, apparatuses described herein may further comprise a product purification zone 210, where one or more product polyol streams are purified to generate one or more high purity polyol streams 220 and 230 by any suitable conventional purification technique.

Depending upon the particulars of the glucose-to-polyol catalyst systems employed in the second reaction zone 60 and third reaction zone 240, second catalyst recovery zone 64 and/or third catalyst recovery zone 242 are optionally utilized to separate catalyst particles from the second effluent 62 and third effluent 242, respectively. In some embodiments, second catalyst particles 66 are separated from the second effluent 62 and/or third catalyst particles 246 are separated from the third effluent 242 either before or after recovery of products and/or co-products. Catalyst particles may be separated from the second effluent 62 and/or third effluent 242 using one or more techniques such as direct filtration, settling followed by filtration, hydrocyclone, fractionation, centrifugation, the use of flocculants, precipitation, extraction, evaporation, or combinations thereof.

While at least one exemplary embodiment has been presented in the foregoing detailed description of the invention, it should be appreciated that a vast number of variations exist. It should also be appreciated that the exemplary embodiments described above are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A method of catalytically generating a polyol from biomass, the method comprising the steps of:
pretreating a feed stream comprising biomass and polymeric lignin to reduce the amount of polymeric lignin in the feed stream;
contacting the pretreated feed stream to acid conditions in a first reaction zone, wherein the acid conditions in the first reaction zone are sufficient to hydrolyze a saccharide from the biomass to generate glucose and comprise an acidic aqueous environment having a pH from about 2.0 to about 6.5, a temperature of about 20° C. to about 350° C. and a pressure of about 15 psig to about 2500 psig;
directing at least a first portion of a first effluent from the first reaction zone to a second reaction zone wherein the composition of the first portion may be the same as the first effluent or a subset thereof; and
contacting the at least first portion of the first effluent with a first saccharide-to-polyol catalyst system under conditions suitable for catalytic conversion of saccharide to polyol.

2. The method of claim 1, wherein contacting the at least first portion of the first effluent with the first saccharide-to-polyol catalyst system comprises contacting the at least first portion of the first effluent with the first saccharide-to-polyol catalyst system in the presence of hydrogen.

3. The method of claim 1, contacting the at least first portion of the first effluent with the first saccharide-to-polyol catalyst system comprises contacting the at least first portion of the first effluent with a first saccharide-to-polyol catalyst system comprising two active metal components.

4. The method of claim 1, wherein contacting the at least first portion of the first effluent with the first saccharide-to-polyol catalyst system under conditions suitable for catalytic conversion of saccharide to polyol comprises contacting the at least first portion of the first effluent with a nickel tungsten carbide catalyst system under conditions suitable to catalytically convert glucose to ethylene glycol.

5. The method of claim 1, wherein contacting the at least first portion of the first effluent with the first saccharide-to-polyol catalyst system comprises contacting the at least first portion of the first effluent with a supported first saccharide-to-polyol catalyst system.

6. The method of claim 1, further comprising directing a second portion of the first effluent from the first reaction zone to a third reaction zone wherein the composition of the second portion may be the same as the first effluent or a subset thereof, and contacting the second portion of the first effluent with a second saccharide-to-polyol catalyst system under conditions suitable for catalytic conversion of saccharide to polyol, wherein the first saccharide-to-polyol catalyst system and the second saccharide-to-polyol catalyst system are different.

7. The method of claim 6, wherein contacting the first portion of the first effluent with the first saccharide-to-polyol catalyst system comprises contacting the first effluent with a saccharide-to-polyol catalyst system that catalyzes a hydrogenation reaction, and wherein contacting the second portion of the first effluent with the second saccharide-to-polyol catalyst system comprises contacting the second portion with a second saccharide-to-polyol catalyst system that catalyzes a saccharide-to-polol reaction and a retro-aldol reaction.

8. The method of claim 6, wherein contacting the first portion of the first effluent with the first saccharide-to-polyol catalyst system comprises contacting the at least first portion of the first effluent with a supported first saccharide-to-polyol catalyst system, contacting the second portion of the first effluent with the second saccharide-to-polyol catalyst system comprises contacting the second portion of the first effluent with a supported second saccharide-to-polyol catalyst system, or both.

9. The method of claim 1, wherein contacting the at least first portion of the first effluent with the first saccharide-to-polyol catalyst system is conducted in an ebullating catalyst bed system, immobilized catalyst reaction system having catalyst channels, augured reaction system, fluidized bed reactor system, mechanically mixed reaction system, or slurry reactor system.

10. The method of claim 6, wherein contacting the first portion of the first effluent with the first saccharide-to-polyol catalyst system and contacting the second portion of the first effluent with the second saccharide-to-polyol catalyst system are independently conducted in an ebullating catalyst bed system, immobilized catalyst reaction system having catalyst channels, augured reaction system, fluidized bed reactor system, mechanically mixed reaction system, or slurry reactor system.

11. The method of claim 1, further comprising pretreating the biomass via sizing, drying, grinding, or a combination thereof into solid particles of a size that may be flowed or moved through a continuous process using a liquid or gas flow, or mechanical processing, hot water treatment, steam treatment, pyrolysis, thermal treatment, chemical treatment, biological treatment, catalytic treatment, or combinations thereof, wherein the step of pretreating the biomass is conducted prior to contacting the feed stream to the acid conditions in the first reaction zone.

12. The method of claim 1, wherein the generated polyol comprises ethylene glycol, propylene glycol, or a combination thereof.

13. The method of claim 1, further comprising generating a second effluent, wherein the second effluent comprises a polyol and a co-product, wherein the polyol comprises ethylene glycol, propylene glycol, or a combination thereof, and the co-product is an alcohol, organic acid, aldehyde, monosaccharide, polysaccharide, phenolic compound, hydrocarbon, glycerol, depolymerized lignin, carbohydrate, or protein.

14. The method of claim 13, further comprising separating the second effluent to generate at least one product polyol stream.

15. A method of generating polyols from biomass, the method comprising the steps of:
contacting a feed stream comprising biomass to acid conditions in a first reaction zone, wherein the acid conditions in the first reaction zone are sufficient to hydrolyze a saccharide from the biomass to generate glucose;
directing a first portion of a first effluent from the first reaction zone to a second reaction zone;
contacting the first portion of the first effluent with a first saccharide-to-polyol catalyst system under conditions suitable for catalytic conversion of saccharide to polyol and generating a first product polyol stream, wherein the first saccharide-to-polyol catalyst system comprises a saccharide-to-polyol catalyst system that catalyzes a hydrogenation reaction;
directing a second portion of the first effluent from the first reaction zone to a third reaction zone; and
contacting the second portion of the first effluent with a second saccharide-to-polyol catalyst system under conditions suitable for catalytic conversion of saccharide to polyol and generating a second product polyol stream, wherein the second saccharide-to-polyol catalyst system comprises a saccharide-to-polyol catalyst system that catalyzes a retro-aldol reaction;
wherein the first product polyol stream and the second product polyol stream are different, and the composition of polyols catalytically generated from the biomass is based on the relative amounts of first effluent directed to the second and third reaction zones.

16. The method of claim 15, further comprising pretreating the biomass via sizing, drying, grinding, or a combination thereof into solid particles of a size that may be flowed or moved through a continuous process using a liquid or gas flow, or mechanical processing, hot water treatment, steam treatment, pyrolysis, thermal treatment, chemical treatment, biological treatment, catalytic treatment, or combinations thereof, wherein the step of pretreating the biomass is conducted prior to contacting the feed stream to the acid conditions in the first reaction zone.

17. An apparatus for catalytic generation of a polyol from biomass, the apparatus comprising:
a first reaction zone configured to receive a feed stream comprising biomass, subject the feed stream to conditions suitable for acid catalyzed hydrolysis of a saccharide in the feed stream to generate glucose, and provide a first effluent comprising glucose, wherein the first reaction zone is not configured to receive a hydrogen stream; and
a second reaction zone in fluid communication with the first reaction zone, the second reaction zone configured to receive and contact at least a portion of the first effluent with a hydrogen stream and a first saccharide-to-polyol catalyst system under conditions suitable for conversion of glucose to a polyol.

18. The apparatus of claim 17, further comprising a pretreatment zone in fluid communication with the first reaction zone and configured to pretreat biomass via one or more of sizing, drying, grinding, or a combination thereof into solid particles of a size that may be flowed or moved through a continuous process using a liquid or gas flow, or mechanical processing, hot water treatment, steam treatment, pyrolysis, thermal treatment, chemical treatment, biological treatment, catalytic treatment, prior to the feed stream being received by the first reaction zone.

19. The apparatus of claim 17, further comprising a third reaction zone in fluid communication with the first reaction zone, the third reaction zone configured to receive and contact a second portion of the first effluent with a hydrogen stream and a second saccharide-to-polyol catalyst system under conditions suitable for conversion of glucose to a polyol, wherein the first and second saccharide-to-polyol catalyst systems are different.

20. The apparatus of claim 19, wherein the first saccharide-to-polyol catalyst system comprises a catalyst that catalyzes a hydrogenation reaction, and the second saccharide-to-polyol catalyst system comprises a catalyst that catalyzes a retro-aldol reaction.
